(19) United States
(12) Patent Application Publication
(10) Pub. No.: US 2024/0024246 A1
(43) Pub. Date: Jan. 25, 2024

(54) STABLE PROLONGED RELEASE FORMULATION OF VITAMIN C AND A PROCESS FOR PREPARATION THEREOF

(71) Applicant: INVENTIA HEALTHCARE LIMITED, Mumbai (IN)

(72) Inventors: Rajat SHAH, Mumbai (IN); Vishal SHAH, Mumbai (IN); Shajahan ABDUL, Madurai (IN)

(21) Appl. No.: 18/257,160

(22) PCT Filed: Aug. 24, 2021
(86) PCT No.: PCT/IB2021/057747

§ 371 Date: Jun. 13, 2023

(30) Foreign Application Priority Data
Dec. 25, 2020 (IN) 202021056476

(51) Int. Cl. A61K 9/20; A61K 31/375; A61K 36/738; A61K 45/06; A61K 33/30

(52) U.S. Cl. CPC A61K 9/2054; A61K 31/375; A61K 9/2068; A61K 9/2013; A61K 36/738; A61K 45/06; A61K 33/30; A61K 9/2095

(57) ABSTRACT

The invention relates to stable prolonged release vitamin C formulation, which is free of any added stabilizer and exhibits prolonged release of the active. The formulation is comprised of at least one hydrophobic non-polymeric excipient and hydrophilic release controlling agent along with at least one excipient. The invention also relates to the process for preparation, wherein the active may be treated with hydrophobic release controlling agent, followed by aqueous granulation with hydrophilic release controlling agent. The granules can be converted into compressible dosage forms like tablets or filled in capsules or sachets. The formulation is stable and releases more than 85% of vitamin C over a period of 8 to 24 hours. The formulation can be suitably orally administered to the subjects in need thereof, for maintaining effective levels of vitamin C over prolonged time.

### FIELD OF THE INVENTION

The invention relates to stable prolonged release vitamin C formulation and a process for preparation thereof. More specifically vitamin C formulation as described herein is free of any added stabilizer and it exhibits prolonged release of the active for more than 8 hours. The formulation is comprised of about 10 to 20% by weight of at least one hydrophobic non-polymeric excipient and about 10 to 40% by weight of hydrophilic release controlling agent. The invention also relates to the process for preparation of stable vitamin C formulation, wherein the active may be treated with hydrophobic release controlling agent at specific temperature conditions. The resulting molten mass is granulated with hydrophilic release controlling agent using process of aqueous granulation. The granules may be mixed with suitable excipient and converted to compressible dosage forms, filled in the sachets or capsules or formulated in the form of jellies or gummies. The formulation is stable and releases more than 85% of vitamin C over a period of 24 hours. The process for preparation is simple, convenient and employs use of commonly used equipment, thus making it time and cost effective. The formulation can be suitably orally administered to the subjects in need thereof, for maintaining significant concentration of vitamin C in blood plasma over prolonged time.

### BACKGROUND

Vitamin C (Ascorbic acid) is a water soluble and thermolabile vitamin, being an essential nutrient for human life. It is known to play an important role as antioxidant due to its presence in the body fluids. It causes an increase in the rate of absorption of iron, calcium and folic acid and hence reduces allergic reactions, boosts the immune system, stimulates the formation of bile in the gall bladder and facilitates the excretion of various steroids. In the body vitamin C plays an essential role in the production of collagen tissue around bones, teeth, cartilage, skin, and damaged tissue. It has shown a prominent pharmacological effect in number of disease conditions such as scurvy, common cold, osteoarthritis, hypertension, heart diseases, cancer, diabetes mellitus, asthma, wound healing, pregnancy, gout and eye diseases. Because of all these favourable effects, it has been used in a variety of cosmetic and pharmaceutical formulations.

Vitamin C is highly soluble in water and alcohol and is easily oxidised to dehydroascorbic acid in its solubilised form. The rapid degradation in aqueous media is still a major factor in the formulation of its products. It is also reported that its oxidation occurs rapidly in an alkaline environment especially at higher temperatures (>50° C.) and its reaction with oxygen is strongly catalysed by metal ions, particularly cupric and ferric ions. The degradation proceeds both by aerobic and anaerobic pathways and depends upon many factors such as oxygen, temperature, light, pH and storage conditions. To achieve maximum stability of vitamin C in various foods, cosmetics and pharmaceutical formulations, different strategies have been employed in the industry. These include the use of stabilizers such as citric acid, vegetable oil, antioxidants, synergists, emulsifiers and the like. The techniques of entrapment of vitamin C in multiple emulsions and encapsulation in nanosuspension have shown significant improvement in the stabilization. The control of medium pH, polarity and viscosity also prolong the shelf life of vitamin C.

Although Vitamin C is a substance of extreme importance to the body, this vitamin is not synthesized by humans. Therefore, to meet daily needs, it is necessary to eat foods rich in vitamin C or intake of medicines containing ascorbic acid as an active ingredient. To maintain saturation level of this substance in the body, the recommended daily dose of ascorbic acid varies from several mg up to 1-2 g, according to the nutritional scientists. However, ascorbic acid in aqueous media undergoes rapid oxidation under natural light, thermal and alkaline conditions, resulting in its decomposition and the loss of its biological activity. Therefore, the search for stable dosage forms that can release ascorbic acid for an extended period and in controlled manner is of paramount importance.

Controlled or sustained release drug formulations became very popular in the last two decades, since they overcome some of the disadvantages of conventional solid dosage forms such as fluctuations of the drug concentration in the plasma and on the site of action, frequency of dosing, drug toxicity and costs. After administration, controlled release dosage forms are designed to provide continuous supply of small but therapeutic amounts of active compound at rates sufficiently controlled to maintain therapeutic plasma concentration levels and prolong therapeutic action over desired time span.

IN patent application 201301108 discloses oral modified release composition as a dietary supplement which comprises of pulverized ascorbic acid, vegetable oil and non-swelling release retardant like wax or fatty acid. Tocopherol and citric acid are used as stabilizers in the formulation. The combination of oil and non-swelling release retardant provides modified release of ascorbic acid from the formulation.

CN patent application 1582922 discloses a vitamin C sustained-release pill composition comprising core of vitamin C, antioxidant, anti-oxidation synergistic agent and the pill is coated with slow-release coating material, plasticizer, pore-forming agent and antifoaming agent.

EP patent application 1942875 discloses a controlled release formulation comprising active agent, e.g. Acerola vitamin C, along with non-polymeric release retardant combined with pH independent non-swelling release retardant (e.g. polyvinyl alcohol, polyvinyl acetate, mixture of polyvinyl acetate (8 parts w/w) and polyvinylpyrrolidone (2 parts w/w) (Kollidon® SR), Polymethacrylic acid derivatives.

JP patent application 06647902 discloses a sustained release oral granule composition comprising ascorbic palmitate in the matrix system of HPMC and polyglyceryl fatty acid ester.

U.S. Pat. No. 8,920,837 relates to the combination of non-swelling pH dependent and non-swelling pH independent release retardant for ascorbic acid formulation.

KR patent application 1826994 covers vitamin C tablet formulation with controlled release property, comprising HPMC of various viscosity grades in combination, along with other excipients such as diluent, binder and lubricant.

US 20150141503 relates to solid, colour-stable L-ascorbic acid compositions, which are in the form of a powder or granule. These improved compositions have a high amount of vitamin C and they have excellent colour stability. The formulation comprises a specific lubricant and suitable binder, which enhances the colour-stability (especially when used in a tablet).

Prior art literature demonstrates variety of approaches for controlling the release of active from the formulation, such as use of pH dependent and pH independent polymers or fatty excipients in combination with coating excipients. However, the release of active from these formulations is not consistent and many of these exhibit dose dumping, thus affecting the overall performance of the formulation. Further, none of these formulations address stability aspect or improved shelf life of the formulations; or they make use of speciality excipient from the category of stabilizers to achieve stability, thus adding to the cost of the product.

### SUMMARY

Developing stable and prolonged release dosage form of vitamin C, which will provide continuous supply of small amounts of the active moiety is an unmet need identified by the team of researchers of the present invention. Such formulations should also ensure longer shelf life and availability of the active form of vitamin C in the body for absorption, to maintain significant concentration in the blood plasma for prolonged time.

The researchers of the present invention have surprisingly found that optimum selection of combination of hydrophobic non-polymeric excipient and the hydrophilic release controlling agent has resulted in stable formulation which exhibits prolonged release of vitamin C over a period of more than 8 hours. The formulation is stable, even though it is free of any added stabilizer and prepared by exposing the active to moderate conditions of heat followed by aqueous granulation. The formulation is granular in nature, which can be compressed in suitable dosage forms, filled in capsules or sachets, or formulated in the form of jellies and gummies for convenient administration to the subjects for maintaining significant concentration of vitamin C in body over 24 hours.

The invention also relates to the process for preparation of stable prolonged release vitamin C formulation, wherein vitamin C may be treated with at least one hydrophobic non-polymeric excipient and the resulting mass may be granulated using at least one hydrophilic release controlling agent by the process of aqueous granulation to get free flowing granules. The granules may be mixed with at least one excipient, which is acceptable in nutraceutical and food industry, for converting into suitable dosage form for oral administration.

The formulation is free of any added stabilizer, but it exhibits stability over a period of proposed shelf life of the product, when stored at standard conditions of temperature and humidity. The formulation, as described herein also exhibits continuous release of more than 85% of vitamin C throughout the day, which helps to maintain significant concentration of the detectable amount of active in the body for prolonged time.

None of the prior art references describe a stabilizer free granular formulation of vitamin C, which is stable, even though prepared by exposing the active to moderately high temperature conditions as well as aqueous granulation process. The formulation is comprised of combination of hydrophobic non-polymeric excipient and hydrophilic release controlling agent. The process of treating vitamin C at specific temperature condition with hydrophobic non-polymeric excipient, and then subjecting it to aqueous granulation using hydrophilic release controlling agent, results into a stable vitamin C formulation with prolonged release of active over desired time.

### Objectives

Main objective of the invention is to provide stable and prolonged release vitamin C formulation and the process for preparation of said formulation.

Another objective of the invention is to provide stable vitamin C formulation, which is free of any added stabilizer.

One more important objective is to provide vitamin C formulation which exhibits prolonged release of more than 85% of the active throughout the day, for more than 8 hours.

Another important objective is to provide vitamin C formulation which exhibits prolonged release of more than 85% of the active, thus resulting into significant concentration of the active in the blood plasma over 24 hours.

Another objective of the invention is to provide stable vitamin C formulation which is comprised of combination of hydrophobic non-polymeric excipient and hydrophilic release controlling agent.

Other objective of the invention is to provide stable formulation comprising about 40 to 75% by weight of vitamin C in combination with about 10 to 20% by weight of hydrophobic non-polymeric excipient and about 10 to 40% by weight of hydrophilic release controlling agent.

Important objective of the invention is to provide stable vitamin C formulation comprising at least one hydrophobic non-polymeric excipient selected from the group of, but not limited to fatty acids, long chain alcohols, fats, lipids, waxes, oils and the combination thereof.

The objective of the invention is also to provide stable vitamin C formulation comprising at least one hydrophilic release controlling agent, selected from the group of cellulose derivatives such as hydroxypropylmethyl cellulose (HPMC), hydroxypropyl cellulose (HPC), ethylcellulose, polyacrylic acid polymers and copolymers, carboxymethylcellulose (CMC), sodium CMC, potassium CMC, calcium CMC, methylcellulose, hydroxyethyl cellulose (HEC), vinyl pyrrolidone-vinyl acetate copolymer, polyvinyl alcohol, starch, carbomer, gums like xanthan gum, guar gum, acacia, locust bean gum, alginates, or mixtures thereof.

One important objective of the invention is to provide a process for preparation of stable vitamin C formulation, wherein the active may be treated with about 10 to 20% by weight of hydrophobic non-polymeric excipient at specific temperature conditions. The resulting mass is granulated with about 10 to 40% by weight of hydrophilic release controlling agent using process of aqueous granulation.

Another objective of the invention is to provide stable vitamin C formulation in the form of granules, wherein the granules may be mixed with about 0.5 to 10% by weight of suitable excipients, acceptable in nutraceutical and food industry and converted into final dosage forms like tablets and capsules.

One more objective of the present invention is to provide stable vitamin C formulation, which may further be comprised of one or more bioactives selected from the group of citrus bioflavonoids, zinc citrate, rose hip extract, vitamin D, vitamin B complex, vitamin E, trace minerals, ginger extract, Echinacea extract, turmeric extract, probiotics, essential amino acids and the combination thereof.

The objective of the invention is to provide process for preparation of prolonged release stable vitamin C formulation, which is simple, convenient and employs use of commonly used equipment, thus making it time and cost effective. The formulation can be suitably orally administered to the subjects in need thereof, for maintaining significant concentration of detectable amount of vitamin C in the blood plasma over 8 hours to 24 hours.

### DETAILED DESCRIPTION

The invention relates to stable prolonged release vitamin C formulation, which is free of any added stabilizer. The formulation may be comprised of at least one hydrophobic non-polymeric excipient and hydrophilic release controlling agent along with one more excipient which is acceptable in nutraceutical and pharmaceutical industry. The invention also relates to the process for preparation, wherein the active may be treated with hydrophobic non-polymeric excipient at specific temperature condition, followed by aqueous granulation with hydrophilic release controlling agent to get the granular formulation. The granules are stable, even though prepared by exposure to conditions like moderate heat and the aqueous granulation process. The stable granules may be mixed with suitable excipient and converted into compressible dosage forms, filled in sachets, capsules or formulated in jellies and gummies.

The formulation as described herein employs vitamin C (also commonly and synonymously called as ascorbic acid and ascorbate interchangeably) which is in the form of white or almost white crystalline powder or colourless and odourless crystal. It may also exist in the form of sodium ascorbate which is white and odourless solid. It is highly unstable in nature and exhibits discoloration or darkening of white shade, upon exposure to air and moisture. It is freely soluble in water and sparingly soluble in ethanol. Vitamin C undergoes degradation after melting at higher temperature such as 190° C. The active may be obtained from natural source or it may be a synthetic product. As vitamin C is sensitive to heat, air and moisture, it is critical to formulate stable formulations of vitamin C in various dosage forms for oral administration.

As per important embodiment of the invention, the formulation may be comprised of about 30 to 85% by weight of vitamin C. More preferably it may be comprised of about 40 to 75% by weight of vitamin C.

As described herein, it was surprisingly observed that treatment of vitamin C with hydrophobic non-polymeric excipient at specific temperature conditions resulted in the form, which was stable even when exposed to accelerated stability conditions of 40° C./75% RH. The unique feature of vitamin C formulation of the present invention being it is free of any specific added stabilizer and also avoids addition of overages, thus reducing the cost of production.

The terminology ‘hydrophobic non-polymeric excipient’ as used herein relates to the formulation component which is used for treating vitamin C at specific temperature condition. This component is found to be explicitly responsible for stabilization of the active, and therefore no other stabilizer is required to be added in the formulation. These carriers are insoluble in water and therefore highly hydrophobic in nature. The hydrophobic non-polymeric excipient may be preferably obtained from natural source, although the carriers may be available from synthetic and semi-synthetic sources.

Treatment of vitamin C with hydrophobic non-polymeric excipient at specific temperature condition results into protection of the active from undergoing degradation while exposing to aqueous environment during granulation phase, followed by hot air during drying. Vitamin C stabilized by treatment with hydrophobic non-polymeric excipient imparts hydrophobicity to the active, which may retard the release of the active from the formulation.

As per one important embodiment, hydrophobic non-polymeric excipient may be selected from the class of, but not limited to lipids, fats, waxes and the combination thereof. The excipient may also be selected from, but not limited to, beeswax, candelilla wax, carnauba wax, spermaceti, paraffin wax, synthetic waxes and the combination thereof.

The hydrophobic non-polymeric excipient may be selected from, but not limited to, saturated fatty acids having 12 to 28 carbons, such as stearic acid, fatty alcohols having from 16 to 44 carbons, cetyl alcohol, pegylated fatty acids, glycerol fatty acid esters, monoglycerides, diglycerides, triglycerides, derivatives of mono-diglycerides, glyceryl behenate, glyceryl monostearate, glyceryl palmitostearate, pegylated vegetable oils, partially hydrogenated oils of soy, cottonseed, palm, sunflower, castor oil, sorbitan esters, polyoxyethylene sorbitan esters, propylene glycol mono- or diesters, phospholipids, phosphatides, cerebrosides, gangliosides, cephalins, lipids, glycolipids, sulfatides, sugar esters, sugar ethers, sucrose esters, sterols, polyglycerol esters, glycerolipid, phosphatic acid, phosphatidylethanolamine, phosphatidylcholine, phosphatidylserine, phosphatidylinositol or other glycerophospholipids, ceramide, sphingolipid, sterol, fat-soluble vitamin, prenol, saccharolipid, polyketide, their salts and esters and the combination thereof.

As per one embodiment of the invention, the formulation as described herein may comprise of about 5 to 30% by weight of hydrophobic non-polymeric excipient. More preferably it may be comprised of about 10 to 20% by weight of this excipient.

The terminology ‘hydrophilic release controlling agent’ as used herein may relate to the formulation component of vitamin C prolonged release formulation, which is hydrophilic and swellable and/or erodible in nature. This is a component which is explicitly responsible for controlling release of vitamin C from the formulation. As described herein, it is used for preparation of granules of vitamin C, which can be conveniently formulated in compressible dosage forms or can be filled in the capsules. Hydrophilic release controlling agent aids to improve compression process by avoiding the problems like picking and sticking, which have been observed during compression of vitamin C granules containing only hydrophobic non-polymeric excipient as release controlling agent.

As per one embodiment of the invention the hydrophilic release controlling agent may be selected from the group of cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), polyacrylic acid polymers and copolymers, vinyl pyrrolidone-vinyl acetate copolymer, polyethylene oxide, polyvinyl alcohol, starch, starch derivatives, modified starch, carbomer, gums like xanthan gum, guar gum, acacia, locust bean gum, alginates, or mixtures thereof.

As per further embodiment, the formulation may be comprised of one hydrophilic release controlling agent or the combination thereof, to achieve prolonged release formulation of the active.

According to one more embodiment of the invention, the formulation may be comprised of about 5 to 50% by weight of hydrophilic release controlling agent, more preferably 10 to 40% of the release controlling agent.

The terminology ‘prolonged release’ as used herein refers to release pattern of the active from the formulation, at desired rate throughout the day over 24 hours. The formulation, as described herein, is designed in such a way that more than 85% of vitamin C is released for a period of more than 8 hours in a day. Thus, specific amount of the active would be released in the body system at definite time intervals for maintaining significant concentration of the active in blood plasma over prolonged time.

As per one embodiment of the invention, prolonged release composition may be comprised of at least one excipient, which is acceptable in nutraceutical, pharmaceutical and cosmetic industry. The excipient may help as a processing aid in formulating the granules in desired dosage form intended for oral administration.

These are commonly used ingredient in industry, and may be selected from the group of, but not limited to, fillers, diluents, lubricants, binders, glidants, anti-caking agents, surfactants, channelizing agents, vehicles, buffers, acidifiers, alkalizers, complexing agents, gum bases, antioxidants, viscosity enhancers, and the combination thereof.

Prolonged release composition of vitamin C as described herein, may be comprised of about 0.5 to 10% by weight of at least one excipient, which is selected from natural, semi-synthetic or synthetic sources.

The formulation may be comprised of diluents known in the art, but not limited to microcrystalline cellulose, silicified microcrystalline, powdered cellulose, microfine cellulose, corn starch, rice bran extract, mannitol, maltodextrin, calcium phosphate, dibasic calcium phosphate, tribasic calcium phosphate, calcium sulfate, or mixtures thereof. The diluents may also be selected from glucose, lactose, sucrose, dextrose, fructose, compressible sugar, or mixtures thereof.

The binders may be selected from the group of low viscosity cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), microcrystalline cellulose; polyvinylpyrrolidone (PVP), vinyl pyrrolidone-vinyl acetate copolymer, polyvinyl alcohol, starch, gums like xanthan gum, guar gum, acacia, locust bean gum, alginates, or mixtures thereof.

The lubricants may be selected from magnesium stearate, calcium stearate, sodium benzoate, talc, or mixtures thereof.

The glidants may be selected from suitable glidants known in the art and commonly used in the industry, selected from the group of stearate, starch, talc and the derivatives.

Therefore, according to important embodiment of the invention, provided herein is a formulation in the form of prolonged release vitamin C, comprising
(a) 40 to 75% of vitamin C, based on the total weight of the formulation,
(b) 10 to 20% of hydrophobic non-polymeric excipient based on the total weight of the prolonged release formulation, selected from the group of fatty acids, long chain alcohols, fats, lipids, waxes, oils and the combination thereof, and
(c) 10 to 40% of hydrophilic release controlling agent, based on the total weight of the prolonged release formulation, selected from the group of cellulose and cellulose derivatives; vinyl pyrrolidone-vinyl acetate copolymer, polyvinyl alcohol, starch, starch derivatives, modified starch, carbomer, gums like xanthan gum, guar gum, acacia, locust bean gum, alginates, or mixtures thereof, and
(d) 0.5 to 10% by weight of at least one excipient, selected from the group of fillers, diluents, disintegrants, lubricants, binders, glidants, anti-caking agents, surfactants, channelizing agents, vehicles, buffers, complexing agents, gum bases, viscosity enhancers and the combination thereof.

According to one more embodiment, the formulation as described herein may also be further comprised of other bioactives such as bioflavonoids, zinc citrate, rose hip extract, vitamin D, B complex and vitamin E, trace minerals, ginger extract, Echinachea extract, turmeric extract, probiotics, essential amino acids and the combination thereof, in combination with vitamin C. The addition of these actives does not affect release profile of vitamin C from the formulation.

These formulations, which can be used as such or in suitable dosage forms, such as compressible dosage forms, capsules, jellies or gummies, may exhibit improved stability when exposed to stress conditions for example at high moisture and/or high temperature conditions.

The formulation as described herein provides reliable, reproducible and consistent prolonged release of vitamin C throughout the day. The formulation is designed in such a way that it will exhibit prolonged release of vitamin C for more than 8 hours in a day in vitro, thus resulting in maintenance of significant concentration of vitamin C in blood plasma over a period of 8 to 24 hours.

As per one more embodiment of the invention, the process for preparation of vitamin C formulation employs commonly available and easy to use industrial equipment.

According to important embodiment of the invention, the process for preparation of the prolonged release formulation may be comprised of treatment of vitamin C with hydrophobic non-polymeric excipient at specific temperature conditions, using suitable equipment, by the way of melt granulation, melt extrusion, melt solidification and the combination thereof. Suitable parameters of temperature, revolutions and torque may be selected for carrying out melt granulation. The process may be carried out by varying the temperature in the range of 40 to 120° C. The molten form can be further processed to get granules suitable for compression into tablet dosage form.

The molten mass may be subjected to aqueous granulation using hydrophilic release controlling agent through process such as fluidized-bed granulation, high-shear granulation, extrusion, or other suitable wet granulation processes. The aqueous granulation process results into uniform and smooth granules, which can be conveniently converted and or compressed in desired dosage form, without any problems of picking or sticking to the equipment. The process for preparation is easy, economic and also makes use of commonly available industrial equipment.

The granules may be formulated in compressible dosage forms, or filled in sachets or capsules using at least one excipient, which is acceptable and commonly used in industry.

The molten mass obtained after treatment of vitamin C with hydrophobic non-polymeric excipient is subjected to stability studies at various temperature and humidity conditions. The granules obtained at this stage are hydrophobic and these may be difficult for subjecting to compression because of picking and sticking problems. The granules are further subjected to aqueous granulation using hydrophilic release controlling agent, which make it suitable for converting into suitable compressible dosage forms.

The prolonged release formulation, as described herein, is subjected to stability study as well as dissolution study to understand release profile of active over extended time period. The formulation is also subjected to evaluation of pharmacokinetic profile in healthy human volunteers to study plasma profile of the active after oral administration in comparison to placebo formulation. The pharmacokinetic evaluation confirmed the prolonged release characteristics of vitamin C formulation over 8 to 24 hours. The data also indicates that the formulation exhibited superiority over the placebo by providing significant blood plasma concentration of Vitamin C for prolonged period over 24 hours.

The following examples serve to illustrate specific embodiments of the invention claimed herein. All percentages are given in relation to the weight and all the temperatures are given in degree Celsius.

### EXAMPLES

### Example 1: Formulation of Vitamin C Prolonged Release Tablets

TABLE 1
Composition of vitamin C prolonged release tablet (Formulation 1)

| Sr. | | |
|---|---|---|
| No | Ingredients | Percentage (%) |
| 1. | Vitamin C | 66.67 |
| 2. | Carnauba wax | 6.67 |
| 3. | Stearic acid | 6.67 |
| 4. | Hydroxypropyl methyl cellulose | 17.66 |
| 5. | Colloidal silicon dioxide | 1.33 |
| 6. | Magnesium Stearate | 1.00 |

| | |
|---|---|
| Total | 100 |

### Process

Vitamin C (Ascorbic acid) was mixed with carnauba wax and stearic acid and the mixture was blended well. The blended dry mix was processed in hot melt extruder at heating temperature 60°−90° C. The resultant mass was milled and passed through mention 30 mesh suitable mesh. The treated mass was granulated with hydroxypropyl methyl cellulose in the ribbon mixer granulator with purified water. The wet granules were dried and sized. The sized granules were lubricated with colloidal silicon dioxide and magnesium stearate. The lubricated granules were compressed into tablets.

### Example 2: Formulation of Vitamin C Prolonged Release Tablets

TABLE 2
Composition of vitamin C prolonged release tablet (Formulation 2)

| Sr. | | |
|---|---|---|
| No. | Ingredients | Percentage (%) |
| 1. | Vitamin C | 60.00 |
| 2. | Stearic acid | 12.00 |
| 3. | Hydroxypropyl methyl | 25.12 |
| | cellulose | |
| 4. | Colloidal silicon dioxide | 1.44 |
| 5. | Magnesium Stearate | 1.44 |

### Process

Vitamin C was mixed with stearic acid and blended well. The blended dry mix was processed in hot melt extruder at heating temperature 60°−90° C. The resultant molten mass was milled and passed through 30 mesh. The treated mass was granulated with hydroxypropyl methyl cellulose in the ribbon mixer granulator with purified water. The wet granules were dried and sized. The sized granules were lubricated with colloidal silicon dioxide and magnesium stearate. The lubricated granules were compressed into tablets using 18×9 mm punches to get the tablets having sufficient hardness with 5.5 mm thickness.

The dissolution of the compressed tablet formulation was carried out in 900 ml citrate buffer of pH 3.0 using paddle at 100 rpm.

Vitamin C released during dissolution study was estimated using HPLC method, using pH 3 buffer and Acetonitrile in a ratio of 3:2 as mobile phase and standard column run conditions. The injection volume was 10 mL and the detection was carried out at 244 nm.

TABLE 3
Release (percent) of vitamin C from prolonged release formulation

| Time (hr.) | Formulation 1 | Formulation 2 |
|---|---|---|
| 1 | 35 | 27 |
| 4 | 64 | 60 |
| 6 | 79 | 74 |
| 8 | 89 | 84 |
| 12 | 102 | 94 |

Formulation 1 and 2 exhibits prolonged release of vitamin C without initial dose dumping and maintains the drug release over a period of more than 8 hours.

### Example 3: Formulation of Vitamin C Prolonged Release Formulation

TABLE 4
Composition of vitamin C prolonged release tablet (Formulation 3)

| Sr. | | |
|---|---|---|
| No. | Ingredients | Percentage (%) |
| 1. | Vitamin C | 70.00 |
| 2. | Carnauba wax | 7.00 |
| 3. | Stearic acid | 7.00 |
| 4. | Hydroxypropyl methyl cellulose | 14.00 |
| 5. | Colloidal silicon dioxide | 1.00 |
| 6. | Magnesium Stearate | 1.00 |

| | |
|---|---|
| Total | 100 |

Process: Same process as used for Example 1 was used for this formulation.

### Example 4: Formulation of Vitamin C Prolonged Release Formulation

TABLE 5
Composition of vitamin C prolonged release tablet (Formulation 4)

| Sr. | | |
|---|---|---|
| No | Ingredients | Percentage (%) |
| 1. | Vitamin C | 60.00 |
| 2. | Stearic acid | 12.00 |
| 3. | Hydroxypropyl methyl cellulose | 25.12 |
| 4. | Colloidal silicon dioxide | 1.44 |
| 5. | Magnesium Stearate | 1.44 |

| | |
|---|---|
| Total | 100 |

### Process: Same Process as Used for Example 2 was Used for this Formulation

### Release of Vitamin C from Compressed Tablets

The tablet formulations 3 and 4 of vitamin C were subjected to dissolution study for checking release of active and the results are tabulated in Table 6.

TABLE 6
Release of vitamin C from prolonged release tablet formulation

| Time (hr) | Formulation 3 | Formulation 4 |
|---|---|---|
| 1 | 23 | 30 |
| 4 | 46 | 60 |
| 6 | 64 | 80 |
| 8 | 74 | 90 |
| 12 | 91 | 97 |

Formulation 3 and 4 exhibits prolonged release of vitamin C without initial dose dumping and maintains the drug release for more than 8 hours.

### Example 5: Stability Study of Prolonged Release Vitamin C Prototype Formulations

The following prototype formulation trials were carried out using various combinations of hydrophobic non-polymeric excipient and/or hydrophilic release controlling agent. These trial formulations were subjected to 40° C./75% RH for one month to study stability aspects of the formulation and role of formulation components on the stability.

TABLE 7
Prototype comparative formulae of vitamin C
and stability study (all quantities in mg)

| Ingredients | Formula 1 | Formula 2 | Formula 3 | Formula 4 | Formula 5 |
|---|---|---|---|---|---|
| Vitamin C | 100.00 | 100.00 | 100.00 | 100 | 100.00 |
| Stearic acid | NA | 20.00 | 20.00 | NA | NA |
| Carnauba wax | NA | NA | NA | 20.00 | 20.00 |
| Hydroxypropyl | 20.00 | NA | 20.00 | NA | 20.00 |
| methyl cellulose | | | | | |
| Purified water | Qs | NA | Qs | NA | qs |
| Initial Assay | 98.6% | 101.5% | 100.8% | 100.9% | 99.8% |
| Assay after | 92.2% | 99.6% | 98.9% | 100.4% | 98.3% |
| exposed to | | | | | |
| 40° C./75% RH | | | | | |
| for one month | | | | | |

### Process

Ascorbic acid, carnauba wax/stearic acid were dispensed and sifted. The mixture was blended well. The blended dry mix is passed through HME operating with the heating temperature 60°−90° C. The resultant mass was milled and passed through suitable mesh, preferably mesh 30. The mass was granulated with HPMC with purified water (Formula 3 and 5). The wet granules were dried and sized. The granules were subjected to stability study using specific conditions of temperature and relative humidity, 40° C./75% RH for one month. The assay of granules was checked before and after the stability study.

The results indicated that the addition of hydrophobic non-polymeric excipient provided better protection to vitamin C during wet granulation, which was quite evident from the outcome of accelerated stability studies, as indicated in Table 7. The formulations in which the active was treated with either stearic acid or carnauba wax (Formula 3 and 5) were stable even after aqueous granulation with hydrophilic release controlling agent, in comparison with Formula 1, in which the assay was lowered down, because the active was directly exposed to aqueous granulation process, without subjecting to treatment with stearic acid or carnauba wax.

### Example 6: Compressibility and Release Profile of Vitamin C Prototype Formulations

TABLE 8
Comparative prototype formulations of vitamin
C and release profile (all quantities in mg)

| Ingredients | Formula 6 | Formula 7 | Formula 8 | Formula 9 | Formula 10 |
|---|---|---|---|---|---|
| Vitamin C | 500.00 | 500.00 | 500.00 | 500.00 | 500.00 |
| Stearic acid | 100.00 | 100.00 | NA | NA | 100.00 |
| Carnauba wax | NA | NA | 100.00 | 100.00 | NA |
| Hydroxypropyl | NA | 209 | NA | 209 | 209 |
| methyl | | | | | |
| cellulose | | | | | |
| Purified water | NA | qs | NA | Qs | qs |
| Calcium | NA | NA | NA | NA | 16 |
| silicate | | | | | |
| Silicon dioxide | 12.00 | 12.00 | 12.00 | 12.00 | NA |
| Mg Stearate | 12.00 | 12.00 | 12.00 | 12.00 | NA |
| Total (mg) | 624.00 | 833.00 | 624.00 | 833.00 | 825.00 |

Percent Vitamin C release in 900 mL of Citrate buffer pH 3.0, operated with Paddle 100 rpm

| | | | | | |
|---|---|---|---|---|---|
| 1 hr | 57 | 30 | 33 | 25 | 29 |
| 4 hr | 93 | 65 | 61 | 61 | 66 |
| 6 hr | 98 | 78 | 70 | 74 | 81 |
| 8 hr | 97 | 88 | 78 | 86 | 91 |
| 12 hr | 95 | 97 | 87 | 96 | 95 |

### Process

Vitamin C, carnauba wax or stearic acid were sifted and the sifted material is blended well. The blended dry mix is passed through HME operating with the heating temperature 60°−90° C. The resultant mass was milled and passed through suitable mesh. The treated granules were granulated with hydroxypropyl methyl cellulose with purified water (Formula 7, 9 and 10). The wet granules were dried and sized. The sized granules were lubricated with colloidal silicon dioxide and magnesium stearate OR with calcium silicate. The lubricated granules were compressed into tablets by using 18 mm×9 mm punches.

Observation: The compression of hydrophobic vitamin C granules prepared by treating with hydrophobic non-polymeric excipient, retarded the release of vitamin C release from the compressed tablets (Formula 6 and Formula 8). However, it was observed that there was capping and sticking of the granules during compression process. Therefore, it was found that these formulations would not be commercially viable and reproducible. When the granules were further treated with hydrophilic release controlling excipient, such as hydroxypropyl methyl cellulose by process of aqueous granulation (Formulation 7, 9 and 10), the resulting granules were easily compressible into tablet dosage form and there was no capping or sticking during the compression process. These formulations exhibit prolonged release of vitamin C without initial dose dumping and maintains the drug release over a period of more than 8 hours.

### Example 7: Formulation of Vitamin C Prolonged Release Formulation

TABLE 9
Composition of Vitamin C Formulation

| Ingredients | Formulation 5 | Formulation 6 | Formulation 7 | Formulation 8 |
|---|---|---|---|---|
| Vitamin C | 80.13 | 80.13 | 80.13 | 80.13 |
| Stearic acid | 16.03 | NA | NA | NA |
| Carnauba wax | NA | 16.03 | NA | NA |
| Cetyl Alcohol | NA | NA | 16.03 | NA |
| Glyceryl Monostearate | NA | NA | NA | 16.03 |
| Silicon dioxide | 1.92 | 1.92 | 1.92 | 1.92 |
| Mg Stearate | 1.92 | 1.92 | 1.92 | 1.92 |
| Total | 100 | 100 | 100 | 100 |

### Process

Vitamin C was mixed with carnauba wax/stearic acid/cetyl alcohol/glyceryl monostearate and the mixture was blended well. The blended dry mix was processed in hot melt extruder at heating temperature 60°−90° C. The resultant mass was milled and passed through suitable mesh. The treated granules were milled and passed through sieve. The sized granules were lubricated with colloidal silicon dioxide and magnesium stearate or with calcium silicate. The wet granules were dried and sized. The lubricated granules were compressed into tablets by using 18 mm×9 mm punches.

The Vitamin C formulation and their respective dissolution profile are mentioned below

TABLE 10
Dissolution Profile of Vitamin C Composition
Dissolution Apparatus: Paddle
Speed: 100 rpm
Dissolution Medium: Citrate buffer pH 3.0 (900 mL)

Percentage Drug Release Of Formulation

| | | | | |
|---|---|---|---|---|
| Time Interval | 5 | 6 | 7 | 8 |

| | | | | | |
|---|---|---|---|---|---|
| 1 | hr | 57 | 33 | 39 | 18 |
| 4 | hr | 93 | 61 | 79 | 32 |
| 6 | hr | 98 | 70 | 94 | 37 |
| 8 | hr | 97 | 78 | 98 | 43 |
| 12 | hr | 95 | 87 | 98 | 54 |

### Example 8: Formulation of Vitamin C Prolonged Release Formulation

TABLE 11
Composition of Vitamin C Formulation

| Ingredients | Formulation 9 | Formulation 10 | Formulation 11 | Formulation 12 |
|---|---|---|---|---|
| Vitamin C | 60 | 60 | 50.50 | 68.50 |
| Stearic acid | 12 | NA | NA | NA |
| Carnauba wax | NA | 12 | NA | NA |
| Cetyl Alcohol | NA | NA | 10.10 | NA |
| Glyceryl | NA | NA | NA | 13.70 |
| Monostearate | | | | |
| HPMC K4M | 25 | 25 | 37.40 | NA |
| HPMC K100LV | NA | NA | NA | 13.70 |
| Purified water | Qs | qs | Qs | qs |
| Silicon dioxide | 1.50 | 1.50 | 1.0 | 2.05 |
| Mg Stearate | 1.50 | 1.50 | 1.0 | 2.05 |
| Total | 100 | 100 | 100 | 100 |

### Process

Vitamin C was mixed with carnauba wax/stearic acid/cetyl alcohol/glyceryl monostearate and the mixture was blended well. The blended dry mix was processed in hot melt extruder at heating temperature 60°−90° C. The resultant mass was milled and passed through mesh size 30. The treated granules were milled and passed through sieve. The sized granules were lubricated with colloidal silicon dioxide and magnesium stearate or with calcium silicate. The resultant mass was milled and passed through suitable mesh. The treated mass was granulated with hydroxypropyl methyl cellulose with purified water. The wet granules were dried and sized. The lubricated granules were compressed into tablets.

The Vitamin C formulation and their respective dissolution profile are mentioned below

TABLE 12
Dissolution Profile of Vitamin C Composition
Dissolution Apparatus: Paddle
Speed: 100 rpm
Dissolution Medium: Citrate buffer pH 3.0 (900 mL)

Percentage Drug Release of Formulation

| | | | | |
|---|---|---|---|---|
| Time Interval | 9 | 10 | 11 | 12 |

| | | | | | |
|---|---|---|---|---|---|
| 1 | hr | 30 | 25 | 22 | 28 |
| 4 | hr | 65 | 61 | 57 | 64 |
| 6 | hr | 78 | 74 | 71 | 74 |
| 8 | hr | 88 | 86 | 81 | 83 |
| 12 | hr | 97 | 96 | 93 | 93 |

### Example 9 A: Composition of Prolonged Release Vitamin C Formulation Comprising Bioactives

TABLE 13
Vitamin C Formulation

| Ingredients | Formulation 13 | Formulation 14 | Formulation 15 | Formulation 16 | Formulation 17 |
|---|---|---|---|---|---|
| Vitamin C | 60.0 | 70.0 | 60.60 | 56.60 | 57.90 |
| Stearic acid | 12.0 | 7.0 | 12.12 | 11.32 | 11.58 |
| Carnauba wax | NA | 7 | NA | NA | NA |
| HPMC K4M | 25.12 | 14.0 | 25.33 | 23.70 | 24.25 |
| Purified water | Qs | qs | qs | Qs | Qs |
| Rose Hip | NA | NA | NA | 2.26 | NA |
| Extract | | | | | |
| Citrus | NA | NA | NA | 3.40 | NA |
| Bioflavonoids | | | | | |
| Zinc citrate | NA | NA | NA | NA | 3.47 |
| Calcium | NA | NA | 1.95 | NA | NA |
| Silicate | | | | | |
| Silicon dioxide | 1.44 | 1.00 | NA | 1.36 | 1.40 |
| Mg Stearate | 1.44 | 1.00 | NA | 1.36 | 1.40 |
| Total | 100 | 100 | 100 | 100 | 100 |

Ascorbic acid, carnauba wax or stearic acid are sifted and the material is blended well. The blended dry mix is passed through hot melt extruder operating with the heating temperature 60°−90° C. The resultant molten mass is milled and passed through the mesh. The treated granules are granulated with hydroxypropyl methyl cellulose with purified water. The wet granules are dried and sized. The granules are mixed well with citrus bioflavonoids, Rose hip extract or zinc citrate (Formula 15 or 16 respectively). The sized granules are lubricated with colloidal silicon dioxide and magnesium stearate or with calcium silicate. The lubricated granules are compressed into tablets by using 18 mm×9 mm punches for 500 mg strength tablet and using 21.5×11.5 punches for 1000 mg strength.

The dissolution profile for above mentioned Vitamin C formulations is covered in the table below:

TABLE 14
Dissolution Profile of Vitamin C formulation
Dissolution Apparatus: Paddle
Speed: 100 rpm
Dissolution Medium: Citrate buffer pH 3.0 (900 mL)

| | | Percentage Drug Release of Formulation | | | | |
|---|---|---|---|---|---|---|
| Time Interval | | 13 | 14 | 15 | 16 | 17 |
| 1 | hr | 26 | 23 | 29 | 28 | 28 |
| 4 | hr | 62 | 46 | 66 | 62 | 63 |
| 6 | hr | 77 | 64 | 81 | 77 | 78 |
| 8 | hr | 87 | 74 | 91 | 87 | 86 |
| 12 | hr | 94 | 91 | 95 | 96 | 94 |

### Example 9 B

The stability studies were carried out for Formulation 13 and 14 at RH and 25° C./60% RH respectively. The dissolution studies and % Assay testing was performed for 3 months, the details for which are mentioned below in Table 15:

TABLE 15
Dissolution studies and % Assay testing for 3 months

| | | | | | | | | | Vitamin |
|---|---|---|---|---|---|---|---|---|---|
| | | | | | | | | | C |
| | | | | | | | | | Content |
| Sr. | Formulation | | | (% Release) | | | | | % |
| No. | No. | Condition | Month | 1 Hr | 4 Hr | 6 Hr | 8 Hr | 12 Hr | Assay |
| 1 | Formulation | 40° C./75% | Initial | 26 | 62 | 77 | 87 | 94 | 101.99 |
| | 13 | RH | 1 | 28 | 64 | 79 | 89 | 95 | 100.1 |
| | | | 2 | 28 | 63 | 80 | 90 | 96 | 100.6 |
| | | | 3 | 28 | 63 | 79 | 90 | 96 | 100.9 |
| | | 25° C./60% | 3 | 26 | 63 | 81 | 91 | 95 | 102.8 |
| | | RH | | | | | | | |
| 2 | Formulation | 40° C./75% | Initial | 30 | 60 | 80 | 90 | 97 | 102.51 |
| | 14 | RH | 1 | 23 | 46 | 64 | 74 | 91 | 100.8 |
| | | | 2 | 25 | 57 | 71 | 82 | 94 | 100.8 |
| | | | 3 | 32 | 64 | 84 | 92 | 96 | 104.8 |
| | | 25° C./60% | 3 | 30 | 64 | 78 | 87 | 93 | 102.1 |
| | | RH | | | | | | | |

The granules of vitamin C prepared as per above formula of Formulation 13 were subjected to stability study at different storage conditions as shown below and the observation is tabulated in Table 16.

TABLE 16
Stability studies of Bulk granules packed in HDPE drums.

| Formulation | Condition | Month | Granules Assay |
|---|---|---|---|
| Formulation 13 | 40° C./75% RH | Initial | 101.47 |
| | | 1 | 103.6 |
| | | 2 | 100.8 |
| | | 3 | 102.3 |
| | | 6 | 98.7 |
| | 30° C./75% RH | 3 | 100.9 |
| | | 6 | 99.2 |
| | 25° C./60% RH | 3 | 102.5 |
| | | 6 | 99.6 |

It was observed that the granules prepared as per the formula were stable at various storage conditions over the test period of 3 months.

The prolonged release Vitamin C formulation of the invention, as described herein, was found to be stable at two different conditions of temperature and humidity, when stored for 3 months. The formulation also exhibits prolonged release of vitamin C, at initial time point and at the end of stability study. The formulation, as described herein comprised of hydrophobic non-polymeric excipient is stable and exhibits prolonged release of more than 85% of active over a period of more than 8 hours, without any dose dumping. The granules treated with hydrophilic release controlling agent can be conveniently compressed in tablet dosage forms, without any capping and sticking problem.

### Example 10: Evaluation of Pharmacokinetic Profile of Vitamin C Prolonged Release Formulation 13

A double-blind, balanced, randomized, single dose, parallel arm, placebo controlled study was conducted to evaluate the pharmacokinetics of Vitamin C Prolonged Release formulation (called as test product or test formulation) in healthy, adult human subjects under fasting conditions. Another purpose of the study was to monitor the safety and tolerability of a single dose of the test formulation. A total of 18 subjects were allocated for specific treatments as per the randomization schedule (9 subjects in each group). During the study, subjects were fasted for at least 10 hours and administered a single dose of either test product or placebo tablet (look alike of test product, excluding active-vitamin C) as per the randomization.

Blood samples were withdrawn from all 18 subjects at different intervals for characterization of single dose pharmacokinetics of the test formulation. The blood samples were analysed for plasma Vitamin C concentration.

The results are reported in Table 17.

### Plasma Vitamin C—Pharmacokinetic Parameters of Formulation 13

TABLE 17
Pharmacokinetic Parameters (Plasma Vitamin C) - Cₘₐₓ and AUC₀₋₂₄

| | Test Product | | Placebo Tablets | | | |
|---|---|---|---|---|---|---|
| Summary | Cₘₐₓ | AUC₀₋₂₄ | C max | AUC₀₋₂₄ | Test | Placebo |
| Statistics | (ng/mL) | (hr · ng/mL) | (ng/mL) | (hr · ng/mL) | T max (hr) | |
| N | 9 | 9 | 9 | 9 | 9 | 9 |
| Mean | 1396.1965 | 11716.4495 | 179.7678 | 886.6845 | 4.28 | 3.28 |
| SD | 1211.1149 | 10734.7181 | 104.2417 | 273.6267 | 2.53 | 2.66 |
| CV % | 86.74 | 91.62 | 57.99 | 30.86 | 59.06 | 81.12 |
| Min | 439.1245 | 3439.3702 | 59.9866 | 397.8479 | 1.50 | 0.00 |
| Median | 785.3012 | 7242.1116 | 117.4697 | 875.1575 | 4.00 | 1.50 |
| Max | 4215.9999 | 36242.4652 | 352.4596 | 1335.7401 | 8.00 | 7.50 |
| Geometric | 1063.7465 | 8624.7691 | 153.2440 | 843.6681 | 3.61 | — |
| Mean | | | | | | |
| SEM | 403.7050 | 3578.2394 | 34.7472 | 91.2089 | 0.84 | 0.89 |
| p Value | <.0001* | <.0001* | — | | — | — |
| (Test vs | | | | | | |
| Placebo) | | | | | | |

*p-value less than 0.05 (p ≤ 0.05) is statistically significant.

Mean Plasma Vitamin C-Cₘₐₓ (Mean±SD) value for test product and placebo tablets were found to be 1396.1965±1211.1149 ng/mL and 179.7678±104.2417 ng/mL, respectively, (8 times higher than the test product). Statistically significant difference was observed in Cₘₐₓ plasma Vitamin C concentration in test product when compared to placebo (p=<0.0001).

Mean Plasma Vitamin C-AUC₀₋₂₄ (Mean±SD) value for test product and placebo were found to be 11716.4495±10734.7181 hr·ng/mL and 886.6845±273.6267 hr·ng/mL, respectively, (13 times higher with test product). Statistically significant difference in AUC₀₋₂₄ was observed for plasma Vitamin C bioavailability in test product when compared to placebo (p=<0.0001).

For Vitamin C in plasma, the secondary pharmacokinetic parameter Tₘₐₓ was calculated for the test and placebo tablets and the results were compared as shown in Table 17.

Mean Plasma Vitamin C Tₘₐₓ (Mean±SD) for Vitamin C Sustained Release Tablets 500 mg and Placebo Tablet were found to be 4.28±2.53 hr and 3.28±2.66 hr, respectively. The Tₘₐₓ data for the placebo treatment is irrelevant since the Vitamin C concentration obtained with placebo is almost equal to baseline concentration.

The pharmacokinetic evaluation of Vitamin C prolonged release tablets indicates that significant concentration of vitamin C in plasma was found for extended period of time and that confirms the prolonged release characteristics of the test product over 8 to 24 hours. The data also indicates that test formulation showed superiority over the placebo by providing significantly higher plasma concentration of Vitamin C in plasma for prolonged period over 24 hours. The study also concludes that Vitamin C prolonged release formulation, as described herein is well tolerated and safe for administration to the subjects in need thereof.

Vitamin C formulation of the present invention exhibits more than 80% release of the active over 8 hours, as shown through in-vitro dissolution studies and the formulation also produces significant concentration of detectable levels of vitamin C in blood plasma over 24 hours, as tested against the placebo. The formulation is free of any added stabilizer, and it can be suitably orally administered to the subjects in need thereof, for maintaining significant concentration of vitamin C in blood plasma over prolonged time.

1. A stable prolonged release vitamin C formulation, comprising,
to 20% by weight of hydrophobic non-polymeric excipient,
to 40% by weight of hydrophilic release controlling excipient;
to 10% by weight of excipient, acceptable in nutraceutical or pharmaceutical industry;
wherein the formulation is free of any added stabilizer.

2. The stable prolonged release formulation of claim 1, which is comprised of 40 to 75% by weight of vitamin C.

3. The stable prolonged release formulation of claim 1, wherein about 85% vitamin C is released over a period of more than 8 hours.

4. The stable prolonged release formulation of claim 1, wherein the hydrophobic non-polymeric excipient may be selected from the group of fatty acids, long chain alcohols, fats, lipids, waxes, oils and the combination thereof.

5. The stable prolonged release formulation of claim 1, wherein the hydrophilic release controlling agent may be selected from the group of cellulose and cellulose derivatives; vinyl pyrrolidone-vinyl acetate copolymer, polyvinyl alcohol, starch, starch derivatives, modified starch, polyacrylic polymers and copolymers, gums like xanthan gum, guar gum, acacia, locust bean gum, alginates, or mixtures thereof.

6. The stable prolonged release formulation of claim 1, which may be comprised of at least one more excipient selected from fillers, diluents, disintegrants, lubricants, binders, glidants, anti-caking agents, surfactants, channelizing agents, vehicles, buffers, complexing agents, gum bases, viscosity enhancers and the combination thereof.

7. The stable prolonged release formulation of claim 1, which may be further comprised of bioactives selected from the group of bioflavonoids, zinc citrate, vitamin D, rose hip extract, and the combination thereof.

8. Process for preparation of stable prolonged release formulation, wherein
a. Vitamin C is mixed well with 10 to 20% by weight of hydrophobic release controlling agent and the blend is subjected to specific temperature condition
b. The resulting molten mass is cooled, sieved and may be granulated with 10 to 40% by weight of hydrophilic release controlling agent using process of aqueous granulation.
c. The resulting granules may be mixed with 0.5 to 10% by weight of at least one pharmaceutically or nutraceutically acceptable excipient to convert into dosage forms;
wherein the process does not make use of any added stabilizer.

9. The process for preparation of claim 8, wherein vitamin C may be treated with hydrophobic release controlling agent at temperature conditions ranging from 50 to 100 degree celsius.

10. The process for preparation of claim 8, wherein the granules may be formulated in compressible dosage form, filled in sachets or capsules, or formulated in jellies or gummies for administration to the subject in need thereof, for maintaining significant concentration of vitamin C in blood plasma over prolonged time.
